(19) United States
(12) Patent Application Publication
(10) Pub. No.: US 2013/0089629 A1
(43) Pub. Date: Apr. 11, 2013

(54) KIT FOR THE TREATMENT OF ONYCHOMYCOSIS BY NITRIC OXIDE

(71) Applicants: Goeran Beijer, Loettorp (SE); Joergen Midander, Stockholm (SE)

(72) Inventors: Goeran Beijer, Loettorp (SE); Joergen Midander, Stockholm (SE)

(73) Assignee: TOPICAL PHARMA AB, Soelvesborg (SE)

(21) Appl. No.: 13/702,666

(22) PCT Filed: Jun. 7, 2011
(86) PCT No.: PCT/SE11/00109

§ 371 Date: Dec. 7, 2012

(30) Foreign Application Priority Data
Jun. 7, 2010 (SE) 1050580-8

(51) Int. Cl. A61K 33/00

(52) U.S. Cl. CPC A61K 33/00
USPC 424/718

(57) ABSTRACT

A kit for the treatment of onychomycosis by nitric oxide, including: a) a pre-treatment part comprising a pharmaceutically acceptable acidifying agent in an amount sufficient to loosen up the superficial outer nail plate layer, b) a treatment part comprising a pharmaceutically acceptable nitrite and at least one polysaccharide or a NO eluting polymer in one container and ascorbic acid in another container in amounts sufficient to produce nitric oxide in an amount that reduces and/or eliminates the onychomycosis upon being mixed and c) at least two devices suitable to apply a) and b) at the treatment site and upon treatment secure that the treatment site is substantially sealed.

### FIELD OF INVENTION

The invention relates to a kit as well as method for the treatment of onychomycosis by nitric oxide.

### BACKGROUND OF INVENTION

There is a big un-met clinical need improving treatment of Onychomycosis especially with locally (topically) applied treatment alternatives.

Current clinical situation can be summarized as follows:
difficult to obtain cure
long term treatment is often required and treatment has to be repeated
local treatments applied topically are intended only in mild cases
oral treatments options are used in more severe cases following failure of local treatments
surgical treatments is considered primitive

Onychomycosis is a fungal infection of the finger and toenails that is caused by dermatophytes, yeasts, or nondermatophyte molds. The primary site from which the infection originates is the bed of the nail (matrix) and the plate under the surface of the nail. The infection damages the nail plate resulting in thickening and discoloration and upon progression, in advanced cases, the nail plate lifts away from the nail bed (onycholysis). This often causes discomfort and is painful.

Onychomycosis is the most common of all diseases of the nails in adults. The incidence is about 5% in the Western World. It is higher in older adults (up to 90% may be affected) and men are more commonly infected than women. Predisposing conditions are chronic diseases (diabetes and circulatory problems) and diseases that suppress the immune system. Overall risk factors include family history, previous trauma to the nails, warm climate, and occlusive or tight footwear.

Current treatment options are either less efficient or associated with side effects that limit usage. Among the options for topical administration amorolfin is provided in a nail lacquer that requires rigorous repeated pre-preparation of the nail and it is not intended for use in case mail matrix is engaged. The lacquer dries to leave a water-insoluble film on top of the nail, which then acts like a drug depot releasing the drug into the nail plate. Terbinafine in a cream or gel is intended for long term treatment.

Griseofulvin and ketoconazole are both intended for systemic (oral) treatment. 10-18 months treatment is recommended for infected toenails. However, relapse rates of 50-85% have been reported. For side effects monthly laboratory monitoring, including liver toxicity, is required.

Itraconazole, terbinafine and fluconazole are more ‘modern’ substances for oral application that upon 6-12 weeks treatment result in somewhat better cure rates and less side effects as compared with grisefulvin and ketoconazole. Still, for purpose of monitoring side effects a complete blood count and liver enzyme tests every 4-6 weeks are required.

So far there is no current single antifungal treatment available that offers a complete cure. Therefore, various combination therapies and/or booster therapies are applied in attempts to improve efficacy. However, these combinations only result in marginal improvement at best.

### SUMMARY OF THE INVENTION

The objective of the present invention is to provide a kit and a method to reduce and eliminate onychomycosis in a mammal for the first time in an efficient and fast way.

In a first aspect the invention relates to a kit for the treatment of onychomycosis by nitric oxide wherein said kit comprises;

a) a pre-treatment part in a carrier, comprising a pharmaceutically acceptable acidifying agent in an amount sufficient to loosen up the superficial outer nail plate layer,

b) a treatment part comprising pharmaceutically acceptable sodium nitrite and at least one polysaccharide or a NO eluting polymer in a container and ascorbic acid in another container in amounts sufficient to produce nitric oxide (NO) that reduces and/or eliminates the onychomycosis upon being mixed, and

c) at least two devices suitable to apply a) and b) at the treatment site and upon treatment secure that the treatment site is substantially sealed, wherein a) and b) will be subsequently applied.

The invention combines a pre-treatment step as well as a treatment step. By a pre-treatment step in which an acidifying agent is applied to the infected site after which a device is applied at the treatment site, wherein said device secures that the treatment site is substantially sealed and that the acidifying agent stays at the treatment site and thereby efficiently can loosen up the superficial outer nail plate layer. After the pre-treatment step the device as well as the acidifying agent is removed prior to a treatment step applied in which at least a mixture of pharmaceutically acceptable sodium nitrite and ascorbic acid and at least one polysaccharide in a liquid formulation, such as water or in a NO eluting polymer is applied to the treatment site. The treatment site is covered by a second device and substantially sealed allowing nitric oxide (NO) to act and reduces and/or eliminates the onychomycosis at the treatment site. The second device is removed after a suitable time period during which the infection was treated by the anti-fungal NO effect. By the invented kit onychomycosis can be treated in an efficient way for the first time. Prolonged treatment which enhances the effectiveness is obtained by having NO released to the critical site in equal amounts over a long period, which is achieved by the polysaccharide or polymer.

In a second aspect the invention relates to a method of treatment of onychomycosis by nitric oxide comprising the steps of

a) applying a pre-treatment step to a treatment site having onychomycosis comprising a pharmaceutically acceptable acidifying agent in an amount sufficient to loosen up the superficial outer nail plate layer in a carrier,

b) applying a device to said treatment site which at least covers the treatment site and secures the treatment site is substantially sealed,

c) allowing said acidifying agent to loosen up the superficial outer nail plate layer,

d) removing a) and b) from said treatment site,

e) mixing sodium nitrite, at least one polysaccharide or a NO eluting polymer and ascorbic acid and applying said mixture to the treatment site,

f) applying a device to said finger/toe which at least covers the treatment site and secures the treatment site is substantially sealed,

g) allowing nitric oxide to reduce and/or eliminate the onychomycosis and

h) removing f) and g) from said treatment site.

The invented kit and method provides for the first time an efficient and fast way to treat onychomycosis, which normally are very difficult to treat completely due to that the infection site is present under the nails and particularly at the root of the nails. By the use of at least one polysaccharide, such as a hydrocolloid, it is for the first time possible to control the release rate of nitrite oxide to the treatment site. The polysaccharider will be dissolved in a liquid such as water upon use and form a network structure. Said network structure provides an optimal release profile of the nitric oxide from the network into the treatment site and thereby deliver an efficient treatment

Further advantages and objects with the present invention will be described in more detail, inter alia with reference to the accompanying drawings.

### BRIEF DESCRIPTION OF THE DRAWINGS

FIG. 1 shows the NO release curves for various concentrations of sodium nitrite and ascorbic acid without addition of starch (EXAMPLE 8).

FIG. 2 shows representative mean NO release curves from 5% (w/w) sodium nitrite and 2% (w/w) ascorbic acid without or with 10% (w/w) starch (EXAMPLE 9).

FIG. 3 shows the NO release over time relationship with various concentrations of sodium nitrite and ascorbic acid in the presence of starch (EXAMPLE 10).

### DETAILED DESCRIPTION OF THE INVENTION

### Definitions

In the context of the present application and invention, the following definitions apply:

The term “acidifying agent” means an agent that has a pH of about 3-5 and/or 4-6 and which has the capacity to loosen up the superficial outer nail plate layer which then enables the possibility for nitric oxide to penetrate and act at the infection site.

The term “pre-treatment” means a step which occur prior to the treatment and which has been finalised prior to that the treatment occur, i.e., two different isolated events. The pre-treatment must be done prior to the treatment step as nitric oxide cannot penetrate the superficial outer nail plate without the nail have been loosened up.

### Embodiments

There are strong data indicating that nitric oxide (NO) is a potential candidate for usage in treatment of onychomycosis. NO is a naturally occurring molecule in the body, endogenously produced, with a large number of biological functions such as relaxation of smooth muscle cells that results in, e.g., dilation of blood vessels and increased intestinal motility, regulation of the cell cycle, nerve transmission and early immune response. The latter means that the molecule has a strong negative effect on pathogens, i.e. bacteria and fungi.

NO is able to pass freely within cells, across cell membranes and between cells as it is an uncharged molecule. It has a messenger role within and between cells without binding to any receptors. NO has a short half-life (within some seconds) and, therefore, its direct effects are transient and local, but difficult to control. It is oxidized to nitrite and nitrate (NO₂⁻ and NO₃⁻) that are stable and inactive.

Overall, for treatment of onychomycosis, topically applied options would seem the preferred choice. As mentioned previously, the primary site from which the fungal infection originates is the bed of the nail (matrix) and the plate under the surface of the nail. However, as the nail plate acts as a strong barrier that hinders the drug to penetrate and disseminate throughout the nail it is of ultimate importance to find a way to circumvent this for purpose of laying the ground for efficient topical treatment of onychomycosis, To apply a highly efficient treatment modality, in terms of antifungal effect in association with limited side effects is vital.

In a first embodiment the invention relates to a kit for the treatment of onychomycosis by nitric oxide wherein said kit comprises;

a) a pre-treatment part comprising a pharmaceutically acceptable acidifying agent in an amount sufficient to loosen up the superficial outer nail plate layer in a carrier,

b) a treatment part comprising pharmaceutically acceptable sodium nitrite and at least one polysaccharide or a NO eluting polymer in a first container and ascorbic acid in a second container in amounts sufficient to produce nitric oxide (NO) in an amount that reduces and/or eliminates the onychomycosis upon being mixed, and

c) at least two devices suitable to apply a) and b) at the treatment site and upon treatment secure that the treatment site is substantially sealed, wherein a) and b) will be subsequently applied.

The pre-treatment part and the treatment part will never come into contact with each other upon use on an infected site, since they are applied subsequently. The polysaccharide and the NO eluting polymer have the same purpose of releasing NO in a controlled manner over an extended time period.

The acidifying agent may be selected from the group consisting of absorbic acid, ascorbyl palmitate, salicylic acid, lactic acid, citric acid, benzoic acid and tartaric acid. One example is salicylic acid. The amount of the acidifying agent may be from about 1 to about 5% w/w, such as 1, 2, 3, 4 or 5% w/w or from about 1 to about 10% w/w, such as 2, 4, 6, 8 or 10% w/w.

During the pre-treatment step the acidifying agent penetrates through which the nail plate, the nail bed and surrounding skin folds are primed prior to the treatment step. The pre-treatment step may be from about one to about 12 hours depending on for example the thickness of the nail, such as 1, 2, 3, 4, 5 6, 8, 9, 10, 11 or 12 hours. The time-interval may vary, i.e. for the initial treatment occasions it may be longer (6-12 hours) while for subsequent repeated treatments it may be reduced to, e.g., 1-6 hours, such as 1, 2, 3, 4, 5, or 6 hours.

The carrier in the above mentioned kit is selected from the group consisting of cream, paste, gel and ointment, lotion, foam, emulsion. Cream with certain viscosity is preferred choice. For the pre-treatment step the acidifying agent is preferably mixed in a cream with certain viscosity. The base could be, for instance, an emulsifying ointment. In one aspect the carrier comprises salicylic acid.

The carrier for the NO-producing sodium nitrite and ascorbic acid is preferably a gel with ceratin viscoelasticity. The carrier to be used with NO is at least one polysaccharide, such as for example a base based on starch molecules that are mixed with water allowing formation of a gel that are soft but not flowing. Alternatively the carrier is a NO eluting polymer.

The treatment step which is the specific anti-fungal treatment step with nitric oxide may be performed from about one to about six hours, such as 1, 2, 3, 4, 5 or 6 hours. The pre-treatment effect results in an increased penetration/diffusion of the subsequently delivered nitric oxide. Thus, NO directly reaches areas of importance for treatment of onychomycosis, i.e. the bed of the nail (matrix) and the plate under the surface of the nail. The amount of sodium nitrite in the kit may be from 1% to about 10% (w/w) such as 1, 2, 3, 4, 5, 6, 7, 8, 9, or 10% and the amount of ascorbic acid from 0.4, to 4% (w/w), such as 0.4, 0.8, 1.2, 1.6, 2, 2.4, 2.8, 3.2, 3.6 or 4% (w/w). Nitric oxide will be applied in doses that are likely to exert an efficient anti-fungal effect, such as between 5 to 1000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the treatment site the concentration may be higher compared to further away from the treatment site.

Use of sodium nitrite alone in combination with, ascorbic acid gives rise to an extreme initial release of nitrite oxide (see EXAMPLE 8). Therefor a new approach was used wherein sodium nitrite was mixed with a polysaccharide, wherein said polysaccharide was solubilised in a liquid such as water prior to use.

The addition of at least one polysaccharide gave rise to a lower initial release and a higher prolonged and stable release of nitrite oxide (see EXAMPLE 9). Thereby a more stable product is achieved and the treatment regime prolonged and improved. The polysaccharide may be any polysaccharide such as a hydrocolloid, such as starch, agar, agarose, carrageenan, alginate, chitosan, pectins, variants or modifications thereof or mixtures thereof, as well as mixtures of various such polysaccharides and also mixture of various types of the same polysaccharide may be used. The polysaccharides may as well be modified in different ways well-known for a person skilled in the art.

A hydrocolloid is defined as a colloid system wherein the colloid particles are dispersed in water. A hydrocolloid has colloid particles spread throughout water, and depending on the quantity of water available that can take place in different states, e.g., gel or sol (liquid). Hydrocolloids can be either irreversible (single-state) or reversible. For example, agar, a reversible hydrocolloid of seaweed extract, can exist in a gel and sol state, and alternate between states with the addition or elimination of heat.

Many hydrocolloids are derived from natural sources. For example, agar-agar and carrageenan are extracted from seaweed, gelatin is produced by hydrolysis of proteins of bovine and fish origins, and pectin is extracted from citrus peel and apple pomace.

Gelatin desserts are made from gelatin powder, another effective hydrocolloid. Hydrocolloids are employed in food mainly to influence texture or viscosity (e.g., a sauce). Hydrocolloid-based medical dressings are used for skin and wound treatment.

Other main hydrocolloids are xanthan gum, gum arabic, guar gum, locust bean gum, cellulose derivatives as carboxymethyl cellulose, alginate and starch.

The important feature being that the polysaccharide acts as a barrier that releases nitric oxide over a prolonged period which increases the treatment period as well as the effect of NO.

If for example starch is used. The starch may be obtained from any kind of starch source such as maize, potato, wheat, rice or cassave. The starch may be modified starch as well as genetically modified starch. The modification may for example be dextrin roasted starch, acid treated starch, alkaline treated starch, bleached starch, oxidized starch, enzyme-treated starch, monostarch phosphate, distarch phosphate, phosphated distarch phosphate, acetylated distarch phosphate, starch acetate, acetylated distarch adipate, hydroxypropyl starch, hydroxypropyl distarch phosphate and starch sodium octenylsuccinate.

The starch may contain various proportions of amylase and amylopectin that stear formation of barrier function. Amylopectin in larger proportions result in formation of gel. Additionally, modification with cross-linking results in that the chains get stuck together into a mesh. All together these assets of the starch applied contribute to more constant release of nitric oxide.

If for example pectin is used. Pectin may be obtained from any sources and may be bought from many chemical industries well-known for a person skilled in the art. Examples of sources include apple, citrus and guavas.

The amount of polysaccharide, such as starch or pectin may be from 1-15% (w/w), such as 2, 3, 4, 5, 6, 7, 8, 9, 10, 11, 12, 13, 14 or 15%. Other examples are shown below under EXAMPLES.

The container of the kit may be selected from the group consisting of ampules, tubes, jars or flasks. The device may be selected from the group consisting of condom, sheath, fingerstall, sock, patch, pad or tape. The device, such as a condom or a fingerstall may be in any suitable size, such as a suitable size for rolling said device over the toe or finger, on which toe or finger the nail to be treated is located. These sizes may for example vary from small, medium, and large sized devices for a little finger, ring finger, middle finger, fore finger, or thumb, or small, medium, and large sized condoms/sheaths for a little toe, the three middle toes, or big toe. The device according to the invention may even have a size suitable for covering a foot or part of a foot, such as a sock. One example being that the carrier is gel, said containers are ampules and said devices are finger stalls.

In another embodiment the part b) is kept in a nitric oxide (NO) eluting polymer. The polymer comprises diazeniumdiolate groups, S-nitrosylated groups, O-nitrosylated groups, or any combinations thereof. The polymer may be selected from the group consisting of amino cellulose, amino dextrans, chitosan, aminated chitosan, polyethyleneimine, PEI-cellulose, polypropyleneimine, polybutyleneimine, polyurethane, poly(buthanediolspermate), poly(iminocarbonate), CarboxyMethylCellulose, polypeptide, polystyrene, poly(vinyl chloride) and polydiemthylsoloxane.

In a third embodiment the invention relates to a method of treatment of onychomycosis by nitric oxide comprising the steps of

a) applying a pre-treatment step a treatment site having onychomycosis comprising a pharmaceutically acceptable acidifying agent in an amount sufficient to loosen up the superficial outer nail plate layer in a carrier,

b) applying a device to said treatment site which at least covers the treatment site and secures the treatment site is substantially sealed,

c) allowing said acidifying agent to loosen up the superficial outer nail plate layer,

d) removing a) and b) from said treatment site,

e) mixing sodium nitrite, at least one polysaccharide and ascorbic acid into a solution or sodium nitrite, a NO eluting polymer and ascorbic acid and applying said mixture to the treatment site,

f) applying a device to said finger/toe which at least covers the treatment site and secures the treatment site is substantially sealed,

g) allowing nitric oxide to reduces and/or eliminates the onychomycosis and

h) removing f) and g) from said treatment site.

The different components used in the method being described in detail above.

Accordingly the kit may contain additional active agents to improve the treatment. Where the agent should be placed in the kit is obvious for a person skilled in the art.

One example of such agents are nail growth promoters, which include but are not limited to minoxidil, minoxidil sulfate, retinoids, cysteine and acetyl cysteine, methionine, glutathione, biotin, finasteride and ethocyn, as well as pharmaceutically acceptable salts of these compounds. The preferred growth promoter are minoxidil, minoxidil sulfate, retinoids, cysteine and acetyl cysteine. The particularly preferred nail growth promoters are 2% minoxidil, 2% minoxidil sulfate, and 0.1% retinol.

Examples of other agents include nutrients, they include but are not limited to vitamins, amino acids, and their derivatives. Examples of such agent include but are not limited to vitamin B complex: thiamine, nicotinic acid, biotin, pantothenic acid, choline riboflavin, vitamin B₆, vitamin B₁₂, pyridoxine, inositol, carnitine; ascorbic acid, ascorbyl palmitate, vitamin A, vitamin K, vitamin E, vitamin D, cysteine and N-acetyl cysteine, herbal extracts, and their derivatives.

Other examples include nail conditioners they include but are not limited to mineral-containing compounds, flavonoids and retinoids. These nail conditioners improve general nail conditions, such as strengthening the nails to prevent nail chipping and cracking, and to beautify the nails. Examples of such agents include but are not limited to calcium pantothenate, calcium carbonate, and calcium gluconate. Examples of retinoids include but not limited to retinol (Vitamin A alcohol), retinal (Vitamin A aldehyde), retinyl acetate, etinyl palmitate, retinoic cid, 9-cis-retinoic acid and 13-cis-retinoic acid. When retinoids are the active agents, the concentration of retinoids is from about 0.01% to about 0.5%, preferably, from about 0.05 to about 0.1%. Examples of flavonoids include but not limited to naringenin, quercetin, catechins (e.g., epigallocatechin gallate), theaflavins, robustaflavone, hinokiflavone, amentoflavone, agathisflavone, volkensiflavone, morelloflavone, rhusflavanone, and succedangeaflavanone.

Following examples are intended to illustrate, but not to limit, the invention in any manner, shape, or form, either explicitly or implicitly.

### EXAMPLES

### Example 1

In a study designed to investigate the acidifying effect on the skin surrounding the finger nail as well as the nail itself a paste comprising salicylic acid at 2% w/w was inserted in a fingerstall. The fingerstall was applied over the top of the finger covering it tightly for about 12 hours. A completely empty stall covering another finger served as control for the same time period

Upon removal of the fingerstalls the finger that had been exposed to salicylic acid showed a different appearance in comparison to the finger that was just covered by the fingerstall. Obviously, the salicylic acid resulted in a softening and loosening up of the skin as it was shrunken, however humid, and with a light pale to red color. Furthermore, the coverage and exposure of salicylic acid to the nail itself also loosened up the superficial outer nail plate layer. Upon scratching the nail surface with a sharp metal edge device, nail fragments (keratinized cells) were easily released in contrast to the nail at the finger covered with an empty fingerstall where the nail plate surface was hard.

### Example 2

To produce NO an acidified nitrite was used as donor. Ascorbic acid was used to reduce sodium nitrite to NO resulting in nitrous acid that was further dissociated forming NO and NO₂. Reduction of the nitrous anhydride, N₂O₂, to NO in parallel to oxidation of ascorbic acid to dehydro-ascorbic acid is central. A mixture of 5% (w/w) sodium nitrite and 2% ascorbic acid in freshly prepared solutions was prepared. From the literature this concentration is known to produce NO lasting for one hour. The curve for the formed level of NO in ppm measured over time shows an initial peak within the first ten minutes followed by a declining production for one hour down to about 16% of the peak ppm-value.

### Example 3

A basal effect of nitric oxide is induction of vasodilatation that results in increased blood flow. Furthermore, it is known from various studies that there is a direct correlation between blood flow and the concentration of NO delivered trans-epidermally (Seabra, et al, 2004). However, as NO diffuses passively and has a very short half-life (within seconds) this effect is exerted entirely local, i.e. at the site to which NO-molecules are able reaching during a time period corresponding to its short life-time.

In a series of experiments blood flow was recorded through laser Doppler imaging (LDI) and monitored color changes from blue-to-green-to-yellow-to-red visualized gradual increase in flow and, furthermore, the localization pattern of the various colors informed about the distribution of NO. The top of one finger was exposed to 12 hours salicylic acid paste (2% w/w) in a fingerstall that was replaced for one hour by a fingerstall comprising 15 mL of a mixture of sodium nitrite (5% w/w) and ascorbic acid (2% w/w) that was generating nitric oxide. This combined treatment resulted in an increased blood flow under the nail (yellow color) as well as a markedly increase in the region of the nail matrix (red color), in comparison to another finger that was treated with the mixture of sodium nitrite and ascorbic acid for an hour but not the active pre-treatment with salicylic acid paste but with an empty fingerstall (yellow area just at the top of the finger). Expectedly, similar result as of the latter was also obtained with no pre-treatment at all followed by treatment with the mixture of nitrite and ascorbic acid applied to a third finger.

The result strongly support that the combined treatment with salicylic acid for pre-treatment followed by nitrite/ascorbic acid-mixture results in a significant deposition of the anti-fungal nitric oxide in the target regions.

### Example 4

Further experiments were done with purpose to study blood flow pattern in relation to various defined settings aimed to serve as appropriate controls to the combined active pre- and main-treatment described in Example 3. Blood flow in the finger tip measured by means of Laser Doppler imaging was performed following application to one finger of salicylic acid at 2% w/w only for 12 hours without any further handling. Placebo (empty fingerstall) was used for pre-treatment of another finger followed by placebo main treatment (empty fingerstall) for 1 hr, i.e. no NO generation. Finally, a third finger was exposed to placebo main treatment without any pre-treatment at all.

A similar pattern of flow, i.e. diffuse areas mainly colored blue and green with some yellow spots, was recorded in all three fingers. There was no sign of increased blood flow similar to the result in Example 3.

### Example 5

Perfusion measures established by the laser Doppler technique in conjunction with the various alternative handlings confirm the images. Table 1 summarizes the mean voltage (V) perfusion measures established directly after finishing the treatments. The combined treatment (active/active), i.e. pre-treatment with salicylic acid at 2% w/w in a finger stall for 12 hours followed by 15 mL of a mixture of sodium nitrite (5% w/w) and ascorbic acid (2% w/w) that was generating nitric oxide for one hour resulted in the highest perfusion (2.58V). All other alternatives of placebo and active resulted in perfusion rates that were lower and in the range of 1.75-2.02V.

Most importantly was the finding that application of the NO-generating mixture with placebo pre-treatment or no pre-treatment at all (1.76V and 2.02V, resp.) did not differ from any of the other treatments with placebo and without the NO-generating mixture (1.82V, 1.99V and 1.75V).

The result confirm strongly that it is the specific combination of salicylic acid for pre-treatment and nitrite/ascorbic acid-mixture for main-treatment that is efficient in terms of directing nitric oxide to the important locations, i.e. the bed of the nail (matrix) and the plate under the surface of the nail.

TABLE 1
Perfusion
Perfusion measures established by laser Doppler technique. The tip of
the fingers was treated with various alternative pre- and main-treatments.
Active/active comprised pre-treatment with salicylic acid at 2% w/w
in a finger stall for 12 hours in combination with 15 mL of a mixture
of sodium nitrite (5% w/w) and ascorbic acid (2% w/w) that was
generating nitric oxide for one hour in the fingerstall. Placebo/active
meant application of an empty fingerstall for 12 hours followed by the
active nitrite/ascorbic acid mixture for an hour. Nill/active comprised
just the active nitrite/ascorbic acid for an hour without any pre-treatment
at all. The others, i.e. active/placebo, placebo/placebo and nill/placebo
were the relevant controls without any active main treatment. The
measurements were established directly after finishing the main
treatment (cf. Example 5).

| Pre-treatment/Main treatment | Perfusion (V, mean) |
|---|---|
| Active/Active | 2.58 |
| Placebo/Active | 1.76 |
| Nill/Active | 2.02 |
| Active/Placebo | 1.82 |
| Placebo/Placebo | 1.99 |
| Nill/Placebo | 1.75 |

### Example 6

An additional study was done to evaluate to what extent the perfusion induced by the combined pre-treatment with salicylic acid followed by nitrite/ascorbic acid main-treatment (cf. Example 5) persisted or if it was just a passing change. Perfusion measurements were recorded by laser Doppler technique after active pre-treatment combined with active main-treatment, i.e. nitric oxide generating, and after active pre-treatment followed by placebo for main-treatment. Finally a completely un-treated finger tip served control. Measurements were done immediately after finishing the main-treatment and 20 minutes thereafter.

The result presented in Table 2 first of all shows again that it is the combined alternative (active/active) that induces a perfusion change, 2.00V in comparison to the controls 1.24 (active/placebo) and 1.33V (nill/nill), respectively. Furthermore, the measures after 20 minutes indicates that the effect of the combined treatments persist and is still higher than the control values.

TABLE 2
Perfusion
Perfusion measures established by laser Doppler technique. The tip of
the fingers was treated with various alternative pre- and
main-treatments. Active/active comprised pre-treatment with salicylic
acid at 2% w/w in a finger stall for 12 hours in combination with 15
mL of a mixture of sodium nitrite (5% w/w) and ascorbic acid (2%
w/w) that was generating nitric oxide for one hour in the fingerstall.
Active/placebo meant application of salicylic acid at 2% w/w in a
finger stall for 12 hours followed by an empty fingerstall for one
hour. Nill/nill was a completely un-treated finger tip. The
measurements were established directly after finishing the main
treatment and 20 minutes thereafter (cf. Example 6).

| | Perfusion (V, mean) | |
|---|---|---|
| Pre-treatment/ | Directly after | 20 mins after |
| Main treatment | treatment stop | treatment stop |
| Active/Active | 2.00 | 1.67 |
| Active/Placebo | 1.24 | 1.16 |
| Nill/Nill | 1.33 | — |

### Example 7

As demonstrated in Example 3 the combined treatment results in an increased blood flow under the nail but, importantly, also in the region of the nail matrix, i.e. the root of the nail. Because the infection site of onychomycosis is present under the nails and particularly at the root of the nails further experiments were done to explore the deposition of nitric oxide to these regions.

Treatments with 15 mL of a mixture of sodium nitrite and ascorbic acid to which modified starch (10% w/w) was added in fingerstalls were applied to the top of fingers for one hour. Sodium nitrite was either at 5%, 7.5 or 10% (w/w) concentration mixed with ascorbic acid at 2, 3 and 4% (w/w) concentration, respectively. The fingers were either pre-treated with salicylic acid paste (2%, w/w) in fingerstalls for 12 hours or not.

Blood flow was recorded through laser Doppler imaging (LDI) directly after finishing treatment, i.e. after one hour exposure to nitric oxide. Considering the maximal perfusion (V) the result (cf. Table 3, below) clearly demonstrates how the pre-treatment with salicylic acid paste allows the generated nitric oxide to penetrate the region of the root of the nails to a significantly larger extent than in the case pre-treatment was not applied.

Comparing mean values for perfusion in the tip of the fingers the result indicates a slight dose-response, i.e. the nitric oxide released from higher concentration of sodium nitrite induces higher perfusion (data not shown). This is valid both in case of pre-treatment and not.

TABLE 3
The maximal perfusion (V) in the tip of finger and the root of nail,
respectively, measured by means of laser Doppler imaging after
one-hour treatment with various concentrations of sodium nitrite and
ascorbic acid mixed with 10% (w/w) modified starch. Treatments were
applied in fingerstalls either after pre-treatment with salicylic paste
for 12 hours or not.

| | | | Difference (%) |
|---|---|---|---|
| | Maximal Perfusion (V) | | between root |
| Treatment | Tip of finger | Root of nail | and tip values |
| 5% sodium nitrite | | | |
| with pre-treatment | 3.11 | 3.94 | +27 |
| without pre-treatment | 2.62 | 1.87 | −29 |
| 7.5% sodium nitrite | | | |
| with pre-treatment | 2.54 | 3.19 | +26 |
| without pre-treatment | 3.41 | 1.86 | −72 |
| 10% sodium nitrite | | | |
| with pre-treatment | 2.92 | 2.77 | −5 |
| without pre-treatment | 2.86 | 1.62 | −43 |

### Example 8

A series of experiments were performed in which the release of NO was measured by an NO-analyzer. Sodium nitrite at various concentrations (2.5, 5, 7.5 and 10% w/w, resp.) was mixed with 2% ascorbic acid and measurements were initiated directly upon adding water.

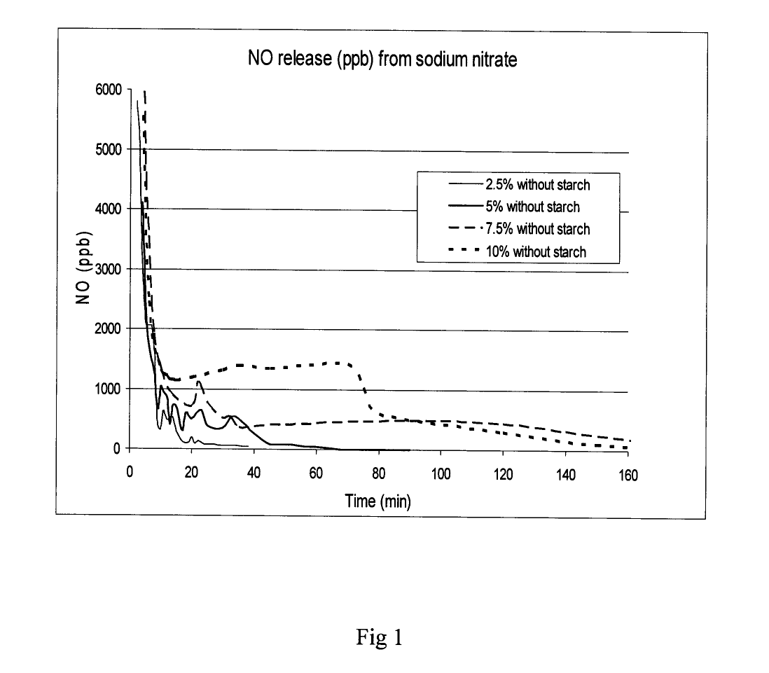

For all concentrations a high initial release (up to about 25000 ppb, not shown) could be measured during the first minutes upon initiation. Thereafter a gradual decrease was noted. For the lower concentrations, i.e. 2.5 and 5% sodium nitrite, the release lasted for about 40-60 minutes while for the higher concentrations (7.5 and 10%) a substantial release (≧400 ppb) continued for up to 90 minutes (FIG. 1).

### Example 9

By adding 10% (w/w) starch prepared from corn (PubChem Substance ID: 24899585, Sigma-Aldrich) to the mixture of sodium nitrite and ascorbic acid a gel was obtained upon addition of water. The aim with this preparation was to reduce the extreme initial release levels and convert this into a more stable release at an appropriate level over longer time.

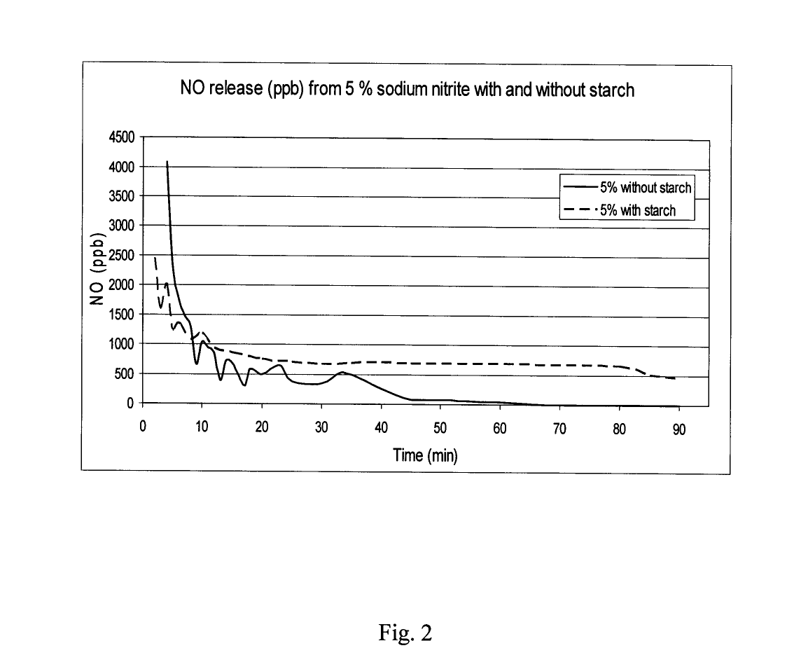

FIG. 2 shows representative mean NO release curves from 5% (w/w) sodium nitrite and 2% w/w) ascorbic acid without or with 10% (w/w) starch. The initial release was reduced from about 19000 ppb (peak level for preparation without starch; not covered in FIG. 2) to about 2500 ppb in the case of sodium nitrite preparation to which starch was added. Furthermore, the release pattern from the preparation with starch was more stabile at a level about 500 ppb for up to 90 minutes.

The addition of starch resulted in a favourable NO release pattern. The starch applied comprises a high content of amylopectin (73%) that upon absorption of water forms a gel. The dense packing of molecules with branched structures formed of amylopectin most likely constitutes a barrier hindering or slowing down the outlet of the nitric oxide.

The gel formation capacity of various concentrations of starch was tested by adding portions to alternate concentrations of sodium nitrite and ascorbic acid according to the following table. One mL of water was added and the appearance of the gel formed was noted directly and after 10 minutes.

| Sodium | Ascorbic | Starch | Gel appearance | |
|---|---|---|---|---|
| nitrite | acid | (%, | | After 10 |
| (%, w/w) | (%, w/w) | w/w) | Directly | minutes |
| 10 | 4 | 10 | Dense, opaque gel | Dense, |
| | | | | opaque gel |
| 10 | 4 | 5 | Less dense, opaque gel | Less dense, |
| | | | | opaque gel |
| 7.5 | 3 | 3.5 | Somewhat watery | Similar |
| | | | but still a gell | |
| 5 | 2 | 2.5 | Watery gel/sauce | Similar |
| 2.5 | 1 | 1.5 | Somewhat wishy- | Similar |
| | | | washy, like a sauce | |

This result indicates that the concentration of starch for purpose being an additive with gel formation capacity must be in the range of 3.5 to 10% (w/w). This doesn't exclude usage of higher concentrations for similar purpose. In the Example referred to above the concentration explored for its barrier effect 10% was used.

### Example 10

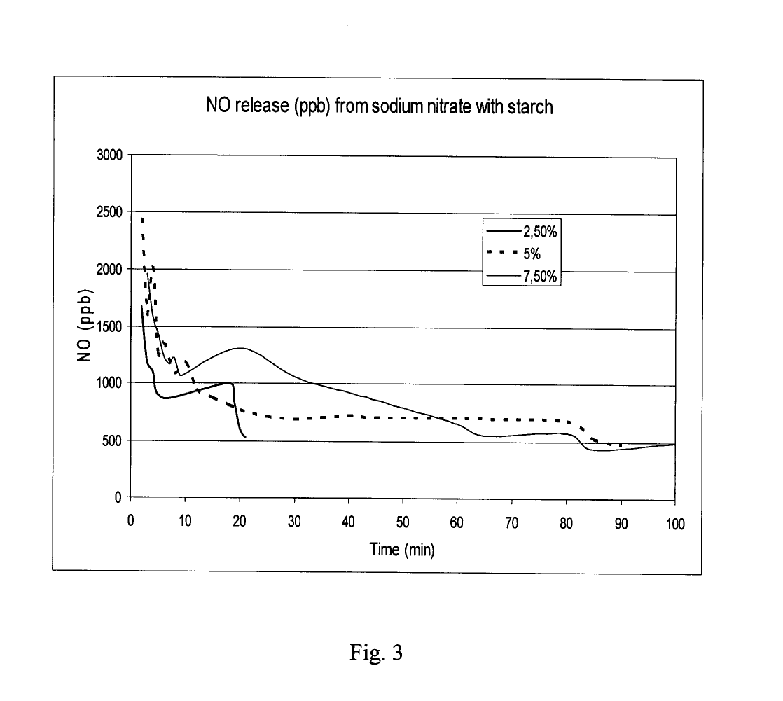

Measurements were done of the NO release in ppb from 2.5, 5 and 7.5% (w/w) sodium nitrite mixed with 1, 2 and 3% (w/w) ascorbic acid, respectively. Starch at a concentration of 10% (w/w) was added to each preparation prior to 1 mL of ionized water. The result indicates a dose related release during the first 50-60 minutes upon initiating the NO-production (FIG. 3).

### Example 11

Similarly to the test with starch presented in Example 9 the capacity of gelatin to form a gel was explored. Regular gelatine (66% oligofructos and 33% food gelatine, Dr Oetker Sweden AB) used for household cooking purposes was dissolved in ionized water at various concentrations and the result was evaluated after 5 and 15 minutes, respectively. The following table summarizes the result.

| Concentration of | Gel appearance | |
|---|---|---|
| gelatine (g/mL) | After 5 minutes | After 15 minutes |
| 0.2 | Watery consistency | Similar |
| 0.4 | Watery consistency | Similar |
| 0.8 | Wishy-washy consistency | Wishy-washy sauce like |
| 1.6 | Semi-opaque fluid | Dense, semi-opaque |
| 2.0 | Opaque fluid | Dense, compact, opaque |

Generally, the formation of gel required some more time in comparison to starch (cf. Example 9), i.e. between 5 and 15 minutes in the case of 1.6 and 2.0 g/mL of the gelatine used. However, the result indicates that gelatin at a titrated concentration, similar to starch, may be used as carrier for the nitric oxide producing substance.

1. A kit for the treatment of onychomycosis by nitric oxide wherein said kit comprises;
a) a pre-treatment part in a carrier comprising a pharmaceutically acceptable acidifying agent in an amount sufficient to loosen up the superficial outer nail plate layer in a carrier,
b) a treatment part comprising pharmaceutically acceptable nitrite and at least one polysaccharide or a NO eluting polymer in one container and ascorbic acid in another container in amounts sufficient to produce nitric oxide (NO) in an amount that reduces and/or eliminates the onychomycosis upon being mixed and
c) at least two devices suitable to apply a) and b) at the treatment site and upon treatment secure that the treatment site is substantially sealed, wherein a) and b) will be subsequently applied.

2. The kit according to claim 1, wherein said acidifying agent is selected from the group consisting of absorbic acid, ascorbyl palmitate, salicylic acid, lactic acid, citric acid, benzoic acid and tartaric acid.

3. The kit according to claim 1, wherein said polysaccharide(s) is a hydrocolloid selected from the group consisting of starch, agar, agarose, carrageenan, alginate, chitosan or pectins, variants or modifications thereof or mixtures thereof.

4. The kit according to claim 1, wherein said carrier is selected from the group consisting of cream, gel, paste and ointment.

5. The kit according to claim 1, wherein said containers are selected from the group consisting of ampules, tubes, jars or flasks.

6. The kit according to claim 1, wherein said devices are selected from the group consisting of condom, sheath, fingerstall, sock, patch, pad or tape.

7. The kit according to claim 1, wherein said carrier is gel, said containers are ampules and said devices are finger stalls and said polysaccharide is starch.

8. The kit according to claim 1, wherein said nitric oxide is in an amount of 1-10% (w/w), said ascorbic acid in an amount of 1-10% (w/w) and said polysaccharide in an amount of 1-15% (w/w).

9. A method of treatment of onychomycosis by nitric oxide comprising the steps of
a) applying a pre-treatment step a treatment site having onychomycosis comprising a pharmaceutically acceptable acidifying agent in an amount sufficient to loosen up the superficial outer nail plate layer in a carrier,
b) applying a device to said treatment site which at least covers the treatment site and secures the treatment site is substantially sealed,
c) allowing said acidifying agent to loosen up the superficial outer nail plate layer,
d) removing a) and b) from said treatment site,
e) mixing sodium nitrite, at least one polysaccharide or a NO eluting polymer and ascorbic acid and applying said mixture to the treatment site,
f) applying a device to said finger which at least covers the treatment site and secures the treatment site is substantially sealed,
g) allowing nitric oxide to reduces and/or eliminates the onychomycosis and
h) removing f) and g) from said treatment site.

10. The method according to claim 9, wherein said acidifying agent is selected from the group consisting of absorbic acid, ascorbyl palmitate, salicylic acid, lactic acid, citric acid, benzoic acid and tartaric acid.

11. The kit according to claim 9, wherein said polysaccharide(s) is a hydrocolloid selected from the group consisting of starch, agar, agarose, carrageenan, alginate, chitosan or pectins, variants or modifications thereof or mixtures thereof.

12. The method according to claim 9, wherein said carrier is selected from the group consisting of cream, gel, paste and ointment.

13. The method according to claim 9, wherein said containers are selected from the group consisting of ampules, tubes, jars or flasks.

14. The method according to claim 9, wherein said devices are selected from the group consisting of condom, sheath, fingerstall, sock, patch, pad or tape.

15. The method according to claim 9, wherein said carrier is gel, said containers are ampules and said devices are finger stalls and said polysaccharide is starch.

16. The method according to claim 9, wherein said nitric oxide is in an amount of 1-10% (w/w), said ascorbic acid in an amount of 1-10% (w/w) and polysaccharide in an amount of 1-15% (w/w).
